(19) United States
(12) Patent Application Publication
(10) Pub. No.: US 2004/0076601 A1
(43) Pub. Date: Apr. 22, 2004

(54) Pharmaceutical form comprising a cell regulating factor and/or a cell proliferation promoter

(76) Inventors: Nicole Bru-Magniez, Paris (FR); Evgueni Myssiakine, Moscow (RU); Sergueevna Myssiakina, Moscow (RU); Alexander Evguenievich Myssiakine, Moscow (RU); Elias Fattal, Paris (FR); Patrick Couvreur, Villebon sur Yvette (FR); Pascal Breton, Tigy (FR)

Correspondence Address: DENNISON, SCHULTZ, DOUGHERTY & MACDONALD, 1727 KING STREET, SUITE 105, ALEXANDRIA, VA, 22314, US

(21) Appl. No.: 10/343,822

(22) PCT Filed: Aug. 6, 2001
(86) PCT No.: PCT/FR01/02554

(30) Foreign Application Priority Data
Aug. 7, 2000 (FR) 00/10396

(51) Int. Cl. A61K 31/74

(52) U.S. Cl. 424/78.31

(57) ABSTRACT

The invention concerns a pharmaceutical form comprising a support material and at least a cell regulating factor and/or a cell proliferation promoter, preferably a growth factor. The invention is characterised in that the support material consists, for at least 90 wt. % of a composition based on methylidene malonate containing 0 to 10 wt. % of one or several methylidene malonate of formula (I) wherein: A and B independently represent a group (a) or (b) wherein R₁ and R₂ independently represent a C₁-C₆ linear or branched alkyl group, and n is an integer ranging between 1 and 5; 10 to 90 wt. %, preferably 50 to 90 wt. % of one or several methylidene malonate oligomer(s) having a molecular weight not more than 6000 and consisting of repeat units of formula (II) wherein A and B are such as defined above; 10 to 90 wt. %, preferably 10 to 50 wt. % of one or several methylidene malonate polymer(s) having a molecular weight more than 6000 and consisting of repeat units of formula (II). Said pharmaceutical form is useful for treating skin lesions.

The invention relates to a novel pharmaceutical form comprising a support material and at least one cell regulation factor and/or cell proliferation promoter.

In the present description the term “support material” is to be understood as meaning a biocompatible polymeric material capable of containing at least one cell regulation factor and/or cell proliferation promoter on its surface or within its structure.

“Cell regulation factors” is to be understood as meaning biochemical mediators which are secreted by cells and are capable of taking part in cellular mechanisms, for example the development, growth or activation of cells, said mediators being especially cytokines, chemokines and growth factors, the latter being particularly preferred. Said cell regulation factors can be natural, synthetic or recombinant.

“Cell proliferation promoter” is to be understood as meaning a natural, recombinant or synthetic molecule which modulates the signaling pathways of cell regulation factors and has an activating effect on cell proliferation, an example being sphingosylphosphorylcholine.

The invention has a valuable application in the field of treatment of skin lesions or dermal and dermo-epidermal wounds for which it is necessary to promote the growth of the cells involved in the healing process, examples being bums, particularly second and third degree bums, ulcers, bedsores, skin ulcers or varicose ulcers.

It also has a valuable application in use as an implant, where the biodegradable support material is capable of gradually releasing cell regulation factor(s), particularly growth factor(s), and/or cell proliferation promoter(s) at the implantation site.

The repair of skin lesions involves a complex series of cellular processes comprising the contraction of the wound, the local accumulation of inflammatory cells, neovascularization, the activation and proliferation of stromal cells (fibroblasts, histiocytes etc.), the synthesis of components of the extracellular matrix (collagen etc.) and epithelialization.

In vivo these processes are regulated by different cytokines, chemokines and eicosanoids.

These include the family of the fibroblast growth factors, or FGFs, of which 22 forms are currently known; these differ in their specificity towards their target cells, and some of them, such as FGF-1 or FGF-2, have the advantage of being capable of regulating the growth of all types of cells involved in the healing of wounds.

Another valuable family is that of the nerve growth factors, or NGFs, which can be involved in the regeneration of nervous, neuromuscular and neuroepithelial tissues.

It is therefore of great interest to have a pharmaceutical form available which enables them to be applied to and kept at the lesion to be treated, while at the same time preserving their stimulating activity on cell growth.

The invention therefore relates to a pharmaceutical form comprising a support material and at least one cell regulation factor and/or cell proliferation promoter, characterized in that at least 90% by weight and preferably at least 95% by weight of the support material consists of a composition based on methylidene malonate containing:

from 0 to 10% by weight of one or more methylidene malonates of formula (I):

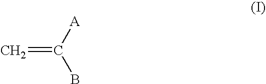

in which:

A and B independently are a group (a) or (b):

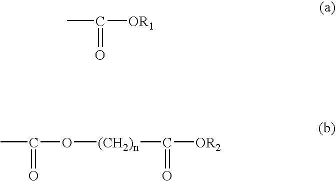

in which R₁ and R₂ independently are a linear or branched alkyl group having from 1 to 6 carbon atoms and n is an integer between 1 and 5;

from 10 to 90% and preferably 50 to 90% by weight of one or more methylidene malonate oligomers having a molecular weight below or equal to 6000 and consisting of repeat units of formula (II):

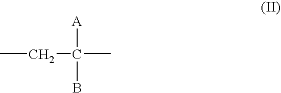

in which A and B are as defined above; and

from 10 to 90% and preferably 10 to 50% by weight of one or more methylidene malonate polymers having a molecular weight above 6000 and consisting of repeat units of formula (II).

“Molecular weight above 6000” is to be understood in particular as meaning a molecular weight above 9000, particularly above 12,000, especially above 25,000, preferably above 50,000 and even of up to 600,000.

In the description, “molecular weight” is to be understood as meaning the weight-average molecular weight, M_{w}, expressed in g/mol of polystyrene (PS) equivalent and measured by gel permeation chromatography (GPC) using a chromatographic apparatus calibrated with standard polystyrene polymers.

According to one preferred feature, in formulae (I) and (II) given above:

A is a group (a) in which R₁ is an ethyl group; and

B is a group (b) in which R₂ is an ethyl group and n is 1.

In one preferred embodiment, at least 90% and preferably at least 95% by weight of the support material consists of a composition based on methylidene malonate containing:

from 50 to 90% by weight of one or more methylidene malonate oligomers having a molecular weight below or equal to 6000, preferably below or equal to 3000, and consisting of repeat units of formula (II); and

from 10 to 50% by weight of one or more methylidene malonate polymers having a molecular weight above 6000.

According to one advantageous feature, the composition based on methylidene malonate contains:

from 55 to 65% by weight of one or more methylidene malonate oligomers having a molecular weight below or equal to 3000, especially of between 300 and 1000, and consisting of repeat units of formula (II); and

from 35 to 45% by weight of one or more methylidene malonate polymers having a molecular weight above 6000, preferably above 9000 and more particularly of between 12,000 and 25,000.

Other advantageous compositions based on methylidene malonate contain:

from 40 to 80% and particularly from 40 to 60% by weight of one or more methylidene malonate oligomers having a molecular weight below or equal to 6000, preferably below or equal to 3000, and consisting of repeat units of formula (II); and

from 20 to 60% and particularly from 40 to 60% by weight of one or more methylidene malonate polymers having a molecular weight above 6000, preferably above 9000 and more particularly of between 12,000 and 25,000.

According to one preferred feature of the invention, the cell regulation factor which can be used for the purposes of the invention is a growth factor. Advantageously, the growth factor(s) can be selected e.g. from fibroblast growth factors (FGFs), transforming growth factor β (TGF-β), platelet-derived growth factor (PDGF), nerve growth factor (NGF) or an association of several of these.

According to one advantageous feature, the growth factor(s) is (are) used on its (their) own or as a complex with a ligand. In fact, the family of the fibroblast growth factors form, with their natural ligands normally present in tissues, such as heparin, heparan sulfate or heparan sulfate proteoglycans, complexes which are very stable under physiological conditions.

It will be preferred to use the basic fibroblast growth factor, also called FGF-2, as a complex with heparan sulfate or with a heparan sulfate proteoglycan.

The FGF-2 and the heparan sulfate can be present in said complex in a molar ratio especially of 1:1 to 1:20 and particularly of 1:1.5 to 1:10.

In the case of a complex with a heparan sulfate proteoglycan, the FGF-2 and the heparan sulfate proteoglycan can be present in said complex in a molar ratio especially of 1:0.01 to 1:0.5.

The preparation of the pharmaceutical form according to the invention comprises incorporating the cell regulation factor(s) or the cell proliferation promoter(s), particularly the growth factor(s), by itself (themselves) or in the form of a complex (complexes) with a ligand, into the support material.

The support material can be prepared by a process in which the desired mixture of monomer(s) and/or oligomer(s) and/or polymer(s) is produced in situ by modifying the reaction conditions.

Alternatively, the support material is prepared by mixing different amounts of monomer(s) and/or oligomer(s) and/or polymer(s), said oligomer(s) and polymer(s) having been prepared beforehand, to give the desired proportion of each of the constituents.

An aqueous solution of the cell regulation factor(s) and/or the cell proliferation promoter(s) is then incorporated into the support material.

Alternatively, if the support material is obtained by mixing different amounts of monomer(s) and/or oligomer(s) and/or polymer(s) prepared beforehand, the aqueous solution of the cell regulation factor(s) and/or the cell proliferation promoter(s) can be incorporated into the support material during one of the steps for the preparation of said support material.

In both cases this incorporation is effected in such a way that the cell regulation factor(s) and/or the cell proliferation promoter(s) do not undergo any denaturation and are present on the surface and/or in the structure of said support material.

The support material according to the present invention is essentially characterized in that it consists mainly of a composition based on methylidene malonate, which itself consists mainly of monomer(s) and/or oligomer(s) having a molecular weight below or equal to 6000 and preferably below or equal to 3000.

According to another advantageous feature of the invention, this composition consists mainly of an oligomer (oligomers) having a molecular weight below or equal to 6000 and a polymer (polymers) having a molecular weight above or equal to 6000.

Such compositions have viscosity and bioadhesion properties which enable them to be used in the treatment of dermal and dermo-epidermal wounds, either by themselves or mixed with other biocompatible components, especially for use of the pharmaceutical form according to the invention as an implant.

In addition, the compositions based on methylidene malonate which can be used within the framework of the invention are biodegradable and release ethanol and glycolic acid, which are generally considered to be non-toxic to the organism at the doses used. This biodegradability is of great value for the release of the growth factor(s) contained especially in the oligomeric or polymeric structure of the support material at the site of the lesion to be treated.

Furthermore, the compositions based on methylidene malonate advantageously make it possible to stabilize the cell regulation factor or cell proliferation promoter in the medium in which it is used, this stability being measurable in particular by the maintenance of their biological activity over time in comparison with the activity of a factor of this type which is free in the medium.

These compositions based on methylidene malonate may easily be prepared by those skilled in the art, optionally by simply mixing its separately prepared constituents (monomeric, oligomeric, polymeric) in an appropriate solvent and subsequently evaporating the solvent off.

Thus the methylidene malonate monomers may be prepared by the process described in patent EP 0 283 346 corresponding to patents U.S. Pat. No. 4,931,584 and U.S. Pat. No. 5,142,098, which are incorporated here by way of reference, after degassing to constant weight under a vane pump vacuum to remove the polymerization inhibitor (SO₂).

The methylidene malonate oligomers and polymers may be synthesized from the aforementioned monomers by an anionic or free radical method.

As regards the preferred compositions based on methylidene malonate, which are formed of a mixture of oligomer(s) and polymer(s), these compositions may also be obtained in a single step; the relative proportions of the constituents may be adjusted by varying the concentration of anionic or free radical initiator in the polymerization medium.

The physicochemical characteristics of the aforementioned compositions based on methylidene malonate may thus easily be adjusted by those skilled in the art to give the support material according to the invention.

In general terms, the constituents of the support material according to the invention other than the aforementioned composition based on methylidene malonate may represent up to 10% by weight of this material.

Of course, these constituents will be chosen so as to form intimate mixtures with the aforementioned compositions based on methylidene malonates.

These constituents may be of a variable nature, being hydrophobic or hydrophilic and of natural or synthetic origin.

The following are examples of such constituents which may be mentioned in particular:

polycyanoacrylates, preferably polyalkyl cyanoacrylates;

polyalkyl methacrylates;

biocompatible polyurethanes;

polyoxyalkylenes;

polyamino acids; and

polyvinyl alcohols.

In general, these constituents will be present in the support material in the form of mixtures with the aforementioned compositions based on methylidene malonate.

It should be noted that, without going outside the framework of the present invention, these constituents may also be present in the support material in the form of monomeric units in random, multiblock or graft copolymers comprising methylidene malonate units of formula (H) as defined above.

These copolymers based on methylidene malonate may be prepared by the conventional polymerization techniques well known to those skilled in the art, among which there may be mentioned polymerization by the anionic method, polymerization by the free radical method or, in the case of multiblock or graft copolymers, the technique of coupling the precursor sequences of the copolymer, these sequences having been appropriately functionalized at the end of the chain beforehand.

These constituents other than the methylidene malonate can also be present in the support material in the form of monomeric units linked to form homopolymers or random, multiblock or graft copolymers devoid of methylidene malonate units of formula (II).

In general, the monomeric units forming the aforementioned constituents will be chosen from the constituent monomeric units of polyacrylates, polysaccharides and polyoxyalkylenes.

Alkyl cyanoacrylates, alkyl methacrylates and itaconates may be mentioned among the constituent monomeric units of polyacrylates which can be used within the framework of the invention.

In general, at least 50% of the monomeric units of the copolymers based on methylidene malonate used within the framework of the present invention will consist of methylidene malonate units.

According to another feature, the invention relates to a pharmaceutical composition comprising a pharmaceutical form as described above and a pharmaceutically acceptable excipient.

The pharmaceutical form according to the invention or the pharmaceutical composition containing it can be administered topically or by injection.

The invention further relates to the use of a pharmaceutical form as defined above for the manufacture of an implant, or for the preparation of a composition to be placed in contact with a wound, or for the preparation of a medicament for the treatment of skin lesions or dermal or dermo-epidermal wounds, for example burns, particularly second and third degree burns, ulcers, bedsores, skin ulcers or varicose ulcers.

It further relates to the use of a pharmaceutical form as defined above for the preparation of a medicament for the regeneration of nervous, neuromuscular or neuroepithelial tissues.

According to another feature, the invention relates to a method of treating skin lesions and dermal or dermo-epidermal wounds, particularly bums, characterized in that it consists in applying, to said lesion or wound, an effective amount of the pharmaceutical form as defined above.

The Examples which follow illustrate the invention without implying a limitation.

### EXAMPLE 1

Preparation of a Pharmaceutical Form According to the Invention Containing an FGF-2/heparan Sulfate Complex

40 mg of methylidene malonate monomer (1-ethoxycarbonyl-1-ethoxy-carbonylmethyleneoxycarbonylethene), kept under low vacuum to remove the polymerization inhibitor (sulfur dioxide) and dissolved in 2 ml of acetone, are introduced into a 25 ml flask. Magnetic stirring is maintained for 15 min.

0.4 ml of 0.1 N NaOH is then added all at once, with continued magnetic stirring.

The magnetic stirring is maintained for about 20 min. A volume of 2.4 ml, containing 40 mg of support material, is obtained. After evaporation of the acetone under vacuum, said material is reduced to ⅕ of its volume.

This gives a support material consisting of a methylidene malonate composition containing about 90% of oligomers having a molecular weight of about 3000 and 10% of polymers having a molecular weight of between about 20,000 and 25,000.

An equal volume of an aqueous solution containing an FGF-2/heparan sulfate complex (hereafter called FGF-2/HS) is added at room temperature.

This complex is prepared beforehand by mixing the 2 components in 100 μl of 5 mM Tris buffer of pH 7.6, the mixture being stirred magnetically for 2 h at room temperature.

The various complexes indicated in Table 1 below were prepared using human recombinant FGF-2 (TEBU, France) and heparan sulfate (SIGMA) purified by electrophoresis, 7.5 kD molecular weight fraction.

TABLE 1

| Complex no. | μg of FGF-2 | μg of heparan sulfate | Molar ratio |
|---|---|---|---|
| 1 | 1 | 0.8 | 1:1.5 |
| 2 | 1 | 3.4 | 1:6.3 |
| 3 | 1 | 5.3 | 1:10 |
| 4 | 1 | 10.7 | 1:20 |

After the addition of the FGF-2/heparan sulfate complex, the mixture is evaporated rapidly to a volume of 0.5 to 0.7 ml and then evaporated under vacuum in an oven overnight until it is completely dry.

This gives 40 mg of a pharmaceutical form according to the invention.

### EXAMPLE 2

Evaluation of the Stimulating Activity of the Pharmaceutical Forms Containing FGF-2/HS Complexes on Cell Proliferation

The pharmaceutical forms according to the invention containing the FGF-2/HS complexes of Example 1 were brought into contact with a conditioned medium, i.e. a culture medium in which the cells have been cultivated for 2-3 days and which therefore contains enzymes secreted by these cells, originating from a culture of Caco-2 human enterocytes (ATCC no. HTB-37).

The pharmaceutical forms are brought into contact with this medium, at a rate of 40 mg of pharmaceutical form per ml of medium, at 37° C. for 24 h. After centrifugation at 500 g for 10 min, the supernatant is introduced into the culture medium of quiescent BP-A31 cells at a rate of 2.5% v/v (5 μl per 200 μl).

The BP-A31 cell line of mouse fibroblasts (chemically transformed 3T3 fibroblasts expressing a high level of FGF receptors—T. Buchou et al., Exp. Cell. Res., 1988, 174(2), 411-420) was used.

The cells are cultivated for 24 h in 24-well plates at a rate of 40,000 cells per well (or 8000 cells per well in 96-well plates) in DMEM (GIBCO) containing 8% of fetal calf serum and antibiotics.

They are then cultivated for 48 h in a medium devoid of serum to give a quiescent culture and to increase their sensitivity to exogenous FGF, before the test samples in the same medium are added.

After incubation for 24 h at 37° C., the proliferation rate is estimated by using the MTT assay (T. Mosmann, J. Immunol. Meth., 1983, 65, 55-63).

MTT (dimethylthiazolyldiphenyltetrazolium bromide) is a yellow dye which, on contact with living cells, is reduced to MTT formazan by the mitochondrial respiratory chain of the cells.

MTT formazan takes the form of blue-violet crystals which, when dissolved, give a coloration proportional to the number of living cells.

The measurement procedure used is as follows:

200 μl of the cellular suspension are placed in each well (NUNCLON DELTA 96-well plates);

incubation is carried out 37° C., air 95%/CO₂ 5%, for a time determined according to the experimental protocol, i.e. 24 h with serum, 48 h without serum, 24 h with products, still without serum;

20 μl of an MTT solution containing 5 mg/ml are added to each well without removing the medium, the MTT stock solution containing 5 mg/ml being prepared in physiological serum and filter-sterilized on a 0.22 μm filter;

incubation is carried out for 2 h 30 min at 37° C., air 95%/CO₂ 5%;

the supernatant is removed by centrifugation at 500 g for 10 min, after which the medium is removed by aspiration; and

200 μl of an extraction solution prepared in the following proportions:

10 g SDS (sodium laurylsulfate)

50 ml DMF (dimethylformamide)

50 mi distilled water

are added to the cells contained in each well.

The pH is adjusted to 4.7 with one drop of acetic acid.

The dishes are shaken for 1 hour or left to stand overnight at 37° C. and a check is carried out under the microscope to ensure that all the crystals of methylformazan have dissolved.

The reading is taken at 570 nm (Labsystems Multiscan MS ELISA plate reader) using reading blanks containing the extraction solution only.

The results, expressed as the percentage proliferation of the cells over 24 h compared with a control culture with no growth factor added, are reported in Table 2 below:

TABLE 2

| Test product | % proliferation |
|---|---|
| pharmaceutical form containing complex no. 1 | 145% |
| pharmaceutical form containing complex no. 2 | 210% |
| pharmaceutical form containing complex no. 3 | 177% |
| pharmaceutical form containing complex no. 4 | 158% |

The results show that the pharmaceutical forms according to the invention stimulate the proliferation of BP-A31 cells.

### EXAMPLE 3

Preparation and Evaluation of Novel Pharmaceutical Forms Containing the FGF-2/HS Complex

40 mg of a material composed of different percentages of methylidene malonate oligomers and polymers prepared from 1-ethoxycarbonyl-1-ethoxy-carbonylmethyleneoxycarbonylethene monomer are introduced into a 20 ml test tube. The material is dissolved in 1 ml of acetone, with constant shaking for 15 minutes. After evaporation of the acetone under vacuum, the material is reduced to ⅕ of its volume, i.e. 200 μl.

An equal volume of an aqueous solution containing an FGF-2/HS complex is added at room temperature. This complex is prepared beforehand as indicated above (Example 1) and the mixture is stirred magnetically for 2 h at room temperature. After the addition of the FGF-2/HS complex, the mixture is evaporated directly under vacuum overnight until it is completely dry. This gives 40 mg of a pharmaceutical form.

Three pharmaceutical forms according to the invention are prepared which vary in the proportion of oligomer/polymer, this variation affecting the viscosity and bioadhesion properties: 40/60 (the most solid form), 60/40 and 80/20 (the most liquid form).

These pharmaceutical forms according to the invention, which vary in the proportion of oligomer/polymer and may or may not contain the FGF-2/HS complexes, are brought into contact with BP-A31 cells according to the protocol described in Example 1 in order to determine the toxicity of the pharmaceutical forms and the biological stimulating activity of the pharmaceutical forms containing the FGF-2/HS complexes on proliferation.

The results given in Table 3 compare the effect of the various pharmaceutical forms, differing in the proportion of oligomer/polymer and containing or not containing FGF-2/HS complex, on proliferation. The results correspond to 3.1% of pharmaceutical forms in the medium. They are expressed as the percentage proliferation of the cells over 24 h compared with a control culture with no support material added.

TABLE 3

| | |
|---|---|
| Ratio of | Proliferation (%) |

| oligomer/polymer % | without FGF-2/HS | with FGF-2/HS |
|---|---|---|
| 40/60 | 109.6 ± 11.5 | 198 ± 8.5 |
| 60/40 | 108.9 ± 9.3 | 201.6 ± 9.8 |
| 80/20 | 105.3 ± 12.6 | 194.7 ± 13.2 |

The results show that FGF-2 formulated in the three pharmaceutical forms according to the invention has a stimulating activity on cell proliferation. Furthermore, in the absence of FGF-2/HS complex, the percentage proliferation is still greater than 100%, showing that the three support materials exhibit no detectable toxicity at the dose used.

### EXAMPLE 4

Effect of Formulating the FGF-2/HS Complex in the Pharmaceutical Forms on the Stability of the Biological Activity of FGF-2

The materials used are on the one hand the three pharmaceutical forms of Example 3, in which the support material consists of different proportions by weight of methylidene malonate oligomers and polymers, and on the other hand free (non-formulated) FGF-2. After storage of the 3 pharmaceutical forms and the free FGF-2 for 7, 14 and 21 days at 4° C., the stimulating activity on cell proliferation is measured by the MTT assay described in Example 2 for 24 h compared with a control culture with no growth factor added.

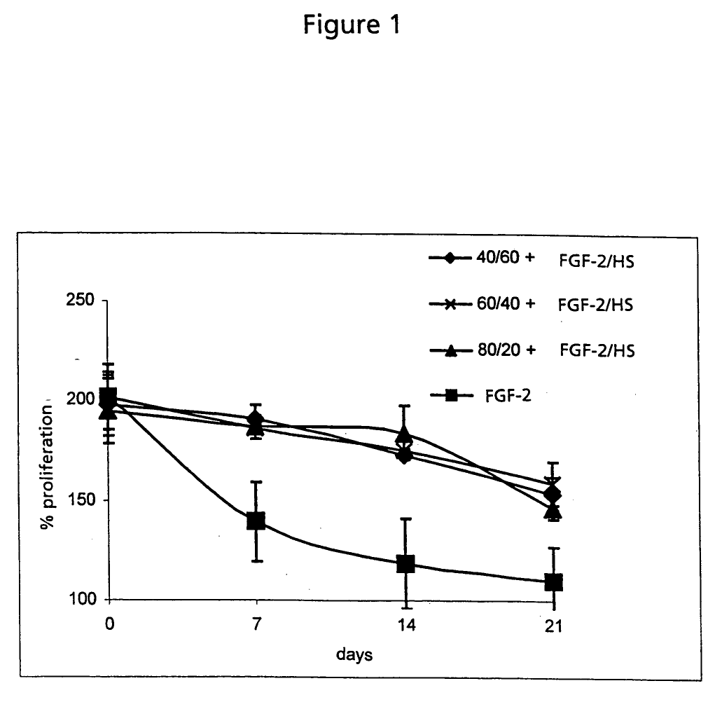

The results are shown graphically in FIG. 1, in which the storage time at 4° C., expressed in days, is plotted on the abscissa and the percentage proliferation is plotted on the ordinate.

The following symbols are used:

♦—represents the pharmaceutical form comprising a support material consisting of 40% of oligomer and 60% of polymer, and the FGF-2/HS complex

x—represents the pharmaceutical form comprising a support material consisting of 60% of oligomer and 40% of polymer, and the FGF-2/HS complex

▴—represents the pharmaceutical form comprising a support material consisting of 80% of oligomer and 20% of polymer, and the FGF-2/HS complex

▪—represents the free (non-formulated) FGF-2

The results show that the three pharmaceutical forms maintain the stimulating activity of FGF-2 on cell proliferation for a period of at least 21 days. By contrast, the activity of free (non-formulated) FGF-2 is comparable to that of FGF-2 formulated in a pharmaceutical form according to the invention only at the earliest time (day zero), said free FGF-2 rapidly losing its biological activity (after 7 days).

### EXAMPLE 5

Preparation and Study of the Stability of Pharmaceutical Forms Containing the Growth Factors PDGF and TGF-β

PDGF and TGF-β were formulated in a material containing 60% by weight of methylidene malonate oligomer and 40% by weight of methylidene malonate polymer prepared from 1-ethoxycarbonyl-1-ethoxycarbonylmethyleneoxycarbonylethene monomer. 100 ng of PDGF or TGF-β were used per 40 mg of material, as described in Example 3. The pharmaceutical form according to the invention, containing or not containing the growth factors, was then brought into contact with L929 fibroblasts according to the protocol described in Example 1 and the stimulating effect of the PDGF or TGF-β on cell proliferation was determined by the MTT assay.

The results, expressed as the percentage proliferation of the cells over 24 h compared with an untreated control culture, are reported in Table 4 below.

TABLE 4

| | | Proliferation |
|---|---|---|
| Factor | Type of formulation | % |
| PDGF | Pharmaceutical form according to the invention | 177.6 ± 3.1 |
| TGF-β | Pharmaceutical form according to the invention | 193.2 ± 3.7 |
| None | 60/40 support material only | 102.4 ± 5.6 |

The results show that, as in the case of FGF-2, the growth factors PDGF and TGF-β both exhibit a stimulating activity on cell proliferation. The support material on its own (without growth factors) exhibits no effect on the cells.

### EXAMPLE 6

Preparation and Study of the Activity of a Pharmaceutical Form Containing the Growth Factor NGF

NGF stimulates the differentiation of neuronal cells, like PC12 rat pheochromocytoma cells or Neuro2A neuroblastomas, at doses similar to those of FGF-2 (between 20 and 100 ng/ml). These cells stop proliferating and produce axonal extensions, which constitutes a morphological criterion indicative of their differentiation.

NGF was formulated in support material containing 60% by weight of methylidene malonate oligomer and 40% by weight of methylidene malonate polymer prepared from 1-ethoxycarbonyl-1-ethoxycarbonylmethyleneoxycarbonylethene monomer (1 μg of NGF formulated per 40 mg of material, as described in Example 2). The pharmaceutical form according to the invention, containing or not containing NGF, was then brought into contact with PC12 and Neuro2A neuronal cells according to the protocol described in Example 1. The stimulating effect on cell differentiation was quantified by determination (counting in a standardized grid) of the percentage of cells exhibiting axonal extensions with a length greater than or equal to 1 cell diameter.

The results, expressed as the percentage of cells exhibiting axons compared: with an untreated culture, are reported in Table 5 below.

TABLE 5

| | PC12 cells with | Neuro2A cells with |
|---|---|---|
| | axons (% of control) | axons (% of control) |
| Pharmaceutical form | 253.3 ± 7.2 | 181.5 ± 3.1 |
| containing NGF | | |
| Free NGF | 290 ± 6.3 | 200.4 ± 4.5 |
| 60/40 support material | 113.2 ± 5.1 | 96.8 ± 3.1 |

The results show that NGF in the pharmaceutical form according to the invention exhibits a biological activity on neuronal differentiation. The support material on its own (without NGF) does not exhibit any effect on the neuronal cells. The pharmaceutical form with NGF therefore represents a means of stimulating neuronal repair.

### EXAMPLE 7

Preparation and Study of the Activity of a Pharmaceutical Form Containing Sphingosylphosphorylcholine (SPC) on Fibroblasts

The metabolite sphingosylphosphorylcholine (SPC) defines a new class of intracellular second messengers exhibiting a broad spectrum of biological activity on the regulation of cell growth and signal transduction. The current literature data on SPC show that it stimulates the proliferation of numerous cell types (fibroblasts, L1 preadipocytes, C6 gliomas, human astrocytes, etc.) which are quiescent or in exponential growth.

SPC was formulated in a support material containing 60% by weight of methylidene malonate oligomer and 40% by weight of methylidene malonate polymer prepared from 1-ethoxycarbonyl-1-ethoxycarbonylmethyleneoxycarbonylethene monomer (100 μg of SPC formulated per 40 mg of material, as described in Example 2). The pharmaceutical form according to the invention, containing SPC or its control solvent, was then brought into contact with BP-A31 fibroblasts according to the protocol described in Example 1. The effect of adding 3.1% to the well, generating a final theoretical SPC concentration of 15 μM, is shown here. The stimulation of cell proliferation was quantified by the MTT assay. The results, expressed as the percentage proliferation of the cells over 24 h compared with an untreated control culture, are reported in Table 6 below.

TABLE 6

| | Proliferation (%) |
|---|---|
| Pharmaceutical form containing SPC | 186.9 ± 9 |
| Free SPC | 158.8 ± 3.2 |
| 60/40 support material | 102.9 ± 9.7 |

The results show that the pharmaceutical form according to the invention containing SPC exhibits a biological stimulating activity on the proliferation of BP-A31 cells. This is comparable to the mitogenic activity of free SPC.

1. Pharmaceutical form comprising a support material and at least one cell regulation factor and/or cell proliferation promoter, characterized in that at least 90% by weight and preferably at least 95% by weight of the support material consists of a composition based on methylidene malonate containing:
from 0 to 10% by weight of one or more methylidene malonates of formula (I): 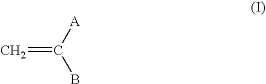
in which:
A and B independently are a group (a) or (b): 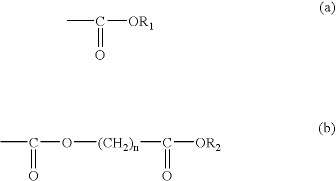
in which R₁ and R₂ independently are a linear or branched alkyl group having from 1 to 6 carbon atoms and n is an integer between 1 and 5;
from 10 to 90% and preferably 50 to 90% by weight of one or more methylidene malonate oligomers having a molecular weight below or equal to 6000 and consisting of repeat units of formula (II): 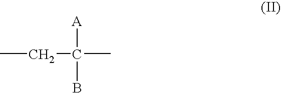
in which A and B are as defined above; and
from 10 to 90% and preferably 10 to 50% by weight of one or more methylidene malonate polymers having a molecular weight above 6000 and consisting of repeat units of formula (II).

2. Pharmaceutical form according to claim 1, characterized in that, in formulae (I) and (II) given above:
A is a group (a) in which R₁ is an ethyl group; and
B is a group (b) in which R₂ is an ethyl group and n is 1.

3. Pharmaceutical form according to claim 1 or 2, characterized in that at least 90% and preferably at least 95% by weight of the support material consists of a composition based on methylidene malonate containing:
from 50 to 90% by weight of one or more methylidene malonate oligomers having a molecular weight below or equal to 6000, preferably below or equal to 3000, and consisting of repeat units of formula (II); and
from 10 to 50% by weight of one or more methylidene malonate polymers having a molecular weight above 6000.

4. Pharmaceutical form according to claim 3, characterized in that the aforementioned composition based on methylidene malonate contains:
from 55 to 65% by weight of one or more methylidene malonate oligomers having a molecular weight below or equal to 3000 and consisting of repeat units of formula (II); and
from 35 to 45% by weight of one or more methylidene malonate polymers having a molecular weight above 6000 and preferably above 9000.

5. Pharmaceutical form according to claim 4, characterized in that the aforementioned composition based on methylidene malonate contains:
from 55 to 65% by weight of one or more methylidene malonate oligomers having a molecular weight of between 300 and 1000 and consisting of repeat units of formula (II); and
from 35 to 45% by weight of one or more methylidene malonate polymers having a molecular weight of between 12,000 and 25,000.

6. Pharmaceutical form according to claim 1 or 2, characterized in that at least 90% and preferably at least 95% by weight of the support material consists of a composition based on methylidene malonate containing:
from 40 to 80% and preferably from 40 to 60% by weight of one or more methylidene malonate oligomers having a molecular weight below or equal to 6000, preferably below or equal to 3000, and consisting of repeat units of formula (II); and
from 20 to 60% and preferably from 40 to 60% by weight of one or more methylidene malonate polymers having a molecular weight above 6000.

7. Pharmaceutical form according to any one of claims 1 to 6, characterized in that the support material comprises up to 10% by weight of one or more constituents other than the aforementioned composition based on methylidene malonate, in the form of monomeric units associated with methylidene malonate units of formula (II) to form random, multiblock or graft copolymers.

8. Pharmaceutical form according to claim 7, characterized in that at least 50% of the monomeric units of said copolymers consist of methylidene malonate units.

9. Pharmaceutical form according to any one of claims 1 to 6, characterized in that the support material comprises up to 10% by weight of one or more constituents other than the aforementioned composition based on methylidene malonate, in the form of monomeric units linked to form homopolymers or random, multiblock or graft copolymers devoid of methylidene malonate units of formula (II).

10. Pharmaceutical form according to any one of claims 1 to 9, characterized in that the cell regulation factor is a natural, synthetic or recombinant growth factor.

11. Pharmaceutical form according to any one of claims 1 to 10, characterized in that the growth factor is selected from fibroblast growth factors (FGFs), platelet-derived growth factor (PDGF), transforming growth factor β (TGF-β), nerve growth factor (NGF) or an association of several of these.

12. Pharmaceutical form according to any one of claims 1 to 11, characterized in that the growth factor(s) is (are) used on its (their) own or as a complex with a ligand.

13. Pharmaceutical form according to any one of claims 1 to 12, characterized in that said growth factor is the basic fibroblast growth factor, or FGF-2, and the ligand is heparan sulfate or a heparan sulfate proteoglycan.

14. Pharmaceutical form according to claim 13, characterized in that the growth factor FGF-2 and the heparan sulfate are complexed in a molar ratio of 1:1 to 1:20 and particularly of 1:1.5 to 1:10.

15. Pharmaceutical form according to claim 13, characterized in that the growth factor FGF-2 and the heparan sulfate proteoglycan are complexed in a molar ratio of 1:0.01 to 1:0.5.

16. Pharmaceutical form according to any one of claims 1 to 15, characterized in that it comprises a natural, synthetic or recombinant cell proliferation promoter.

17. Pharmaceutical form according to claim 16, characterized in that the cell proliferation promoter is sphingosylphosphorylcholine.

18. Pharmaceutical composition containing a pharmaceutical form according to any one of claims 1 to 17 and a pharmaceutically acceptable excipient.

19. Use of a pharmaceutical form according to any one of claims 1 to 17 for the manufacture of an implant.

20. Use of a pharmaceutical form according to any one of claims 1 to 17 for the preparation of a composition to be placed in contact with a wound.

21. Use of a pharmaceutical form according to any one of claims 1 to 17 for the preparation of a medicament for the treatment of skin lesions.

22. Use according to claim 21 for the preparation of a medicament for the treatment of burns, particularly second and third degree bums, ulcers, dermal and dermo-epidermal wounds, bedsores, skin ulcers or varicose ulcers.

23. Use of a pharmaceutical form according to any one of claims 1 to 17 for the preparation of a medicament for the regeneration of nervous, neuromuscular or neuroepithelial tissues.
